(19) United States
(12) Patent Application Publication
(10) Pub. No.: US 2005/0106634 A1
(43) Pub. Date: May 19, 2005

(54) Isolation of neurones and stem cells for nerve fibres from a sample

(76) Inventors: Frank W. Pfrieger, Singen (DE); Karl Naegler, Munchen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/495,361

(22) PCT Filed: Nov. 12, 2002
(86) PCT No.: PCT/EP02/12645

(30) Foreign Application Priority Data
Nov. 12, 2001 (DE) 101-55-130.4

(51) Int. Cl. G01N 33/53; G01N 33/567; C12N 5/08

(52) U.S. Cl. 435/7.2; 435/368; 435/455

(57) ABSTRACT

The invention relates to the use of an L1 promoter-regulated protein, and L1-associated molecule, of L1, or of recognition molecules directed towards the above-mentioned structures, for the isolation of neurons and stem cells for nervous fibers, especially neural and/or neuronal stem cells, from a sample; the invention also relates to a method of isolating neurons and stem cells for nervous fibers using the above-mentioned recognition molecules. The fields of application of this invention are medicine and the pharmaceutical industry.

The invention relates to the use of an L1 promoter-regulated protein, an L1-associated molecule, of L1, or of recognition molecules directed towards the above-mentioned structures, for the isolation of neurons and stem cells for nervous fibers, especially neural and/or neuronal stem cells, from a sample; the invention also relates to a method of isolating neurons and stem cells for nervous fibers using the above-mentioned recognition molecules. The fields of application of this invention are medicine and the pharmaceutical industry.

The central nervous system (CNS) is constituted of various types of cells, with neurons, macroglia cells, such as astrocytes and oligodendrocytes, and microglia cells forming the most important classes. Neurons are important in the electrochemical transmission of information in the nervous system, while microglia cells represent highly important components of the cellular immune response within the CNS, and macroglia cells mainly assume supporting functions (Jacobson, Developmental Neurobiology, New York: Plenum Press, 1991). Macroglia cells form a framework in the CNS, providing neurons with metabolites. More recent investigations have demonstrated that a variety of additional molecular interactions between macroglia cells and neurons take place, which interactions enable CNS development and are also important for the function and plasticity thereof (Kettenmann & Ransom, Neuroglia, Oxford: Oxford University Press, 1995).

Most conveniently, such interactions can be investigated on cultured cells under well-defined conditions. One important precondition for such investigations is to separate the neurons from other cells, so as to allow identification and testing of the direct effect of, in particular, glial, growth and differentiation factors on the neurons. To date, it is merely possible to purify retinal gangliocytes from rats, using a so-called immunopanning procedure (Barres et al., 1998). This procedure utilizes the RGC-specific expression of the Thy-1 cell adhesion protein. Other neurons, particularly CNS neurons, cannot be isolated with the desired purity and yield as yet.

The invention was therefore based on the object of isolating or accumulating neurons and differentiated stem cells, in particular neuronal and/or neural stem cells, from a sample so as to make them useful for various applications, for example.

The invention solves this technical problem by using an L1 promoter-regulated protein, an L1-associated molecule, an L1 cell adhesion protein, a recognition molecule directed towards a protein regulated by an L1 promoter, towards an L1-associated molecule and/or towards an L1 cell adhesion protein for the isolation of neurons and differentiated stem cells, particularly neural and/or neuronal stem cells, from a sample.

Accordingly, the teaching of the invention is based on the surprising finding that neurons and/or differentiated, particularly neural and/or neuronal stem cells can be selected or isolated specifically via the L1 cell adhesion protein, via proteins regulated by the L1 promoter, and via an L1-associated molecule, or via recognition molecules directed towards the above-mentioned structures. In particular, L1 and the above-mentioned L1 equivalents are expressed in the CNS, specifically by neurons. Surprisingly, it has also been possible to demonstrate that not only neurons from the CNS, e.g. from the postnatal CNS, but also from stem cells, especially differentiated stem cells, preferably neuronal differentiated adult or embryonal stem cells, can be isolated via L1 and L1 equivalents. In particular, this is advantageous in those cases where e.g. embryonal stem cells undergo neural differentiation by addition of suitable factors. In addition to neurons, the resulting cell population invariably includes glia cells, and in some cases even other, non-neural types of cells. Such a mixed population is unsuitable particularly in transplantation therapy because non-neuronal, i.e., dividing cells involve the risk of developing into tumors after transplantation. With the aid of the teaching according to the invention it is possible for the first time to obtain a purely neuronal cell population from stem cells, particularly for transplantation purposes, e.g. in neurodegenerative diseases. Furthermore, the neurons thus obtained or isolated can be used to investigate the conditions of neuronal growth and differentiation.

A sample in the meaning of the invention denotes any biological item collected by sampling, or a partial quantity or small amount thereof, the nature of which can be investigated by chemical, biological, clinical means or the like, particularly for the purpose of obtaining neurons or differentiated stem cells from such a sample; for example, the sample can be an accumulation of stem cells or a piece of a brain. More specifically, collecting or obtaining the sample is effected in such a way that the collected partial quantity corresponds to an average of the overall amount. The features determined by investigation or from the sample can be used—e.g. prior to isolating neurons or collecting stem cells—to assess the amount comprised by the sample, thereby allowing conclusions as to the overall amount. For investigation, the samples can be pretreated by mixing, dividing, separating, pre-fractionating, reducing in size, adding enzymes or markers, or by other means. Various ways of pretreating the samples are known to those skilled in the art. Obviously, it may also be envisaged to collect the sample such that it does not correspond to an average of the overall amount. In particular, samples are biological materials comprising structures suitable for transmitting or storing information on a biological basis. For example, these include the CNS or parts thereof, the spinal cord, neocortex, striatum, etc. Furthermore, fluids, cell accumulations or tissues comprising rod, cone and olfactory cells may be concerned, as well as structures comprising amacrinal cells, horizontal cells, bipolar cells, Golgi cells, Purkinje cells, motoneurons, hair, pyramidal and/or basket cells. Accordingly, the sample may comprise numerous other materials such as blood, lymph, urine, cerebrospinal fluid, liquids from bioreactors, lipid mixtures, bone, cartilage, and many others.

Neurons in the meaning of the invention are all nervous cells which, being highly differentiated cells, are capable of receiving, processing and transmitting nervous stimuli. For example, these cells occur in the CNS, in ganglions and in sensory organs and may represent a partial quantity of the sample. Neurons or nervous cells in the meaning of the invention consist of a cell body (=Corpus neuroni) and a nucleus which may be surrounded by a cytoplasm. In the meaning of the invention the cells themselves may be referred to as perikaryon. In the meaning of the invention the nervous cell or neuron also has e.g. neurofibrils, Nissl substance and appendices, as well as one or more dendrites.

For example, the nervous cells can be nervous cells from animals or humans. It is possible, for instance, to isolate adendritic nervous cells or apolar nervous cells which have no appendices, or bipolar nervous cells, as sensitive or sensory nervous cells having one polar dendrite and neurite each, such as occurring in the form of granular cells of the retina, or multipolar nervous cells having several dendrites and a long or short neurite, as motoric cells of the voluntary and autonomic systems. Furthermore, it is possible to isolate polyneuritic nervous cells having a plurality of neurites, e.g. as Cajal cells of the cerebral cortex or pseudo-unipolar nervous cells such as occurring in sensitive ganglions of the head and spinal cord, or unipolar nervous cells wherein two appendices originally present are apparently fused into one, such as occurring in rod and cone olfactory cells, provided these cells can be subsumed under the term neurons in the meaning of the invention. Of course, neuro-secretory nervous cells, as obtained e.g. from the hypothalamus, or neuromelanocytes may also be understood as neurons in the meaning of the invention. Depending on their location in the nervous system, the neurons can also be isolated as preganglionic or post-ganglionic neurons and as interneurons. Other cells defined as nervous cells or neurons according to the invention are, inter alia, amacrinal cells, horizontal cells, bipolar cells, Golgi cells, Purkinje cells, motoneurons, hair, pyramidal and/or basket cells.

Amacrinal cells in the meaning of the invention are multipolar nervous cells, especially in the inner granular layer of the retina of the eye, which cells have short appendices. Horizontal cells are cells which, with their cell bodies containing nuclei, represent interneurons of the optic pathway situated in the inner granular layer of the retina. Horizontal cells may also include the Cajal cells which occasionally occur in the uppermost layer of the cerebral cortex and represent small spindle-shaped nervous cells with long, horizontally oriented appendices. Bipolar cells or bipolar neurons are nervous cells having two appendices extending separately therefrom, and these cells occur in the ganglions of the inner ear and of the retina. Golgi cells in the meaning of the invention are large granular cells of the cerebellar cortex with short neurites or cells to be subsumed under the term Cellulae axiramificatae. Purkinje cells in the meaning of the invention are large, pear-shaped nervous cells in the Stratum gangliosum of the cerebellar cortex, each having two to three dendrites extending vertically into the molecular layer and branching in a plane therein and neurites descending into the cerebellar medulla. The motoneuron is understood to be the last neuron in the efferent innervation of the skeletal muscles, consisting of a ganglion cell situated in the anterior horns of the spinal cord and of the neurite innervating the nervous cells. The motoneuron forms a motoric unit of the skeletal muscle and consists of an alpha-motoneuron with all muscle fibers innervated by this neuron, each muscle fiber having only one motoric end plate. Hair cells are understood to be auditory cells of the Corti apparatus. They are secondary sensory cells between the supporting cells of the Corti organ of the inner ear, provided with auditory hairs. They terminate synaptically at the dendrites of the bipolar ganglion cells of the Ganglion spirale. Pyramidal cells in the meaning of the invention are pyramid-shaped multipolar nervous cells of the cerebral cortex, the apical dendrites thereof extending into the molecular layer, while the shorter ones branch horizontally from the basal corners, and the neurites thereof extending from the cell basis towards the medulla. Basket cells in the meaning of the invention are asteroid nervous cells in the molecular layer of the little brain, the abundant branches thereof, formed by their neurites, braiding as basket fibers around the Purkinje cells.

In the meaning of the invention, nervous cells are particularly those cells occurring in the CNS, and they can be referred to as neurons. According to the invention, however, the occurrence of these neurons is not restricted to the CNS. For example, it is also possible that e.g. basket cells are modified by suitable factors to form cells having neuron properties, in particular.

In addition to neurons or nervous cells or neurocytes, the teaching of the invention can also be used to isolate or separate or accumulate differentiated stem cells, e.g. neural and/or neuronal stem cells, from a sample. Inter alia, neurons, oligodendrocytes and astrocytes may be formed from the adult neural stem cells, especially from the central nervous system. In humans, for example, these cells are predominantly found in the ventricular and subventricular zone of the brain and in the subgranular zone of the Gyros dentatos in the hippocampus. The embryonal stem cells, for example, derive from the early phase of development of an embryo prior to nidation, namely, from the so-called inner cell mass, i.e., the blastocyst. These cells are pluripotent, that is, they are capable of differentiating into any tissue of the human organism. Unlike zygotes, however, they are not capable of developing into complete embryos. Similarly, these stem cells are capable of differentiating into neurons in the meaning of the invention, thereafter being neurons in the meaning of the invention. However, they can be understood as such even prior to differentiation. To use the above-mentioned stem cells e.g. in therapy, it is important that the cell preparation has high purity. With the aid of the teaching of the invention it is possible for the first time to separate the developing neurons from the insulin-producing cells or blood cells developing simultaneously from the embryonal stem cells. The neurons obtained in this way can be used in transplantations, e.g. in Parkinson and Alzheimer patients.

Also, neuronal stem cells in the meaning of the invention are all those cells that are known as stem cells from the nervous system or can be found therein. In the stem cell formation zone approximately one out of three hundred cells is a stem cell. For example, a cell population with 80% purity can be obtained from an adult brain using automatic flow cytometry to provide an almost completely pure population of neuronal stem cells with the aid of the teaching of the invention, provided the stem cells exhibit L1, L1-associated structures or fragments or equivalents of such a structure, e.g. L1 homologues such as neuroglian.

Furthermore, the stem cells obtained, especially neuronal stem cells, can be stimulated—by means of factors known to those skilled in the art—in a way so as to form astrocytes and neurons or nervous cells; in this process the simultaneous occurrence of muscle cells or other cells in the culture can never be excluded. The cells thus obtained can be separated, likewise using the method according to the invention, because—in the course of the individual development of the cells—those cells developing as neurons can be separated from those having e.g. glia cell properties or those exhibiting properties of astrocytes. The neuronal stem cells can be isolated from most various regions of the adult CNS, e.g. from the ventricular walls or from the striatum. Furthermore, they can be obtained from the outer germinal zone of the little brain of newborn mice. Other sources from which neuronal stem cells can be isolated, or stem cells which allow differentiation into neuronal stem cells, are well-known to those skilled in the art.

Accordingly, stem cells in the meaning of the invention are both embryonal and adult stem cells which, in particular, are already neural or differentiated in a neuronal fashion, or allow differentiation to this effect. Surprisingly, not only neurons but also differentiated embryonal and adult stem cells, especially neuronal-differentiated stem cells, can be isolated via L1 or L1 equivalents. L1 equivalents in the meaning of the invention are all those structures interacting with specific recognition molecules in a way so as to allow isolation of neurons and differentiated stem cells. On the one hand, cell adhesion proteins L1 are understood to be neuronal cell adhesion molecules pertaining to the Ig superfamily in vertebrates and invertebrates. In addition to L1, the neuronal cell adhesion molecules—as is well-known to those skilled in the art—include axonin, neuroglian, N-CAM, fascilin, DM-GRASP BEN SC1, IRRI C-RST, the polio virus receptor and others, which are closely related to L1 and therefore can be understood as L1 equivalents in the meaning of the invention. Homologies between cell adhesion molecules of invertebrates and vertebrates, particularly on the level of the secondary and tertiary structures, e.g. between neuroglian and L1 and also, between fascilin and N-CAM and between the IRRS C gene product and DM-GRASP, are well-known to those skilled in the art. In addition to these structural common features of neuronal cell adhesion molecules, i.e., in addition to the L1 equivalents in the meaning of the invention, these moleculee are also capable of significantly promoting neurite growth. L1 in the meaning of the invention is particularly effective by stimulating in particular the neurite growth on Schwann cells by homophilic binding mechanisms between Schwann cells and neurons. Lesions of peripheral nerves give rise to increased expression of L1 especially in neurons and Schwan cells, and L1 may also be associated with axonal regeneration. L1 equivalents in the meaning of the invention may also include all proteins regulated by an L1 promoter. All those proteins or lipid or carbohydrate structures associated with the L1 protein in a way so as to allow isolation of the above-mentioned stem cells and neurons are L1-associated molecules in the meaning of the invention. Those skilled in the art are familiar with the fact that L1-associated molecules may also be sugar or lipid structures, for example, which associate with, e.g. bind to, L1 or L1 equivalents in a way so as to allow the use thereof in the isolation of neurons, neural and/or neuronal stem cells from a sample. Those skilled in the art are familiar with the fact that in addition to L1 and L1 equivalents or L1-associated molecules, it is also possible to use molecules having a close relationship to neuroglian or L1, provided they occur in neurons or in the above-mentioned stem cells. For example, these include the vertebrate molecule contactin wherein the number of FN III domains is merely reduced by one. The above-mentioned L1 equivalents are preserved over a long period of time during the evolution of the nervous system, and therefore, a variety of molecules, especially proteins, but also lipids or carbohydrates associated therewith, are available to those skilled in the art, which can be used in the meaning of the teaching of the present patent application. Obviously, L1 may also be a component of a fusion protein.

In the meaning of the invention, isolation means that the collected neurons or nervous cells or neuronal and/or neural stem cells can be obtained with a purity of 40%, preferably 50%, more preferable 60%, and especially preferably 70, 80, 90, 95, 97, 99 and more than 99.5%. Various methods of isolating cells and determining the degree of purity are well-known to those skilled in the art.

In the meaning of the invention it is of course also possible to use recognition structures directed towards L1, L1-associated molecules or proteins regulated by the L1 promoter, i.e., all L1 equivalents in a broader sense, to isolate neurons and/or the above-mentioned stem cells. For example, immobilization of the recognition molecules, e.g. an antibody against the cell adhesion protein L1, on a culture dish or in a chromatography column filled e.g. with nylon wool, can be envisaged. Now, when contacting a cell suspension or a sample including the neurons or the above-mentioned stem cells with said culture dish or chromatography column, the structures to be isolated can interact with the recognition molecules such that all those cells lacking the above-mentioned L1 equivalents can be separated by washing or other methods known to those skilled in the art from the neurons or stem cells to be isolated.

In a preferred embodiment of the invention the recognition molecule is an antibody, an aptamer, a lectin, an antisense construct, a selective chelator, fragments and/or a combination thereof. Antibody denotes a polypeptide encoded substantially by an immunoglobulin gene or by immunoglobulin genes or fragments thereof specifically binding and recognizing an analyte, i.e., L1, L1-associated molecules, proteins regulated by an L1 promoter, that is, L1 equivalents. Well-known immunoglobulin genes include the kappa-, lambda-, alpha-, gamma-, delta-, epsilon- and mu-genes for the constant region, as well as the innumerable genes for the variable immunoglobulin region. For example, antibodies can be intact immunoglobulin or a number of well-characterized fragments produced by cleavage with various peptidases. Antibody also denotes modified antibodies (e.g. oligomers, reduced, oxidized and labelled antibodies). The term antibody as used in the present specification also encompasses antibody fragments produced either by modification of whole antibodies or by de novo synthesis using recombinant DNA techniques. The term antibody encompasses both intact molecules and fragments thereof, such as Fab, F(ab′)2 and Fv, capable of binding the epitope determinant. In these fragments the auto-antibody's capability of selectively binding its antigen or the L1 equivalent is retained in part, the fragments being defined as follows:
(1) Fab, the fragment which contains a monovalent antigen-binding fragment of an antibody molecule, can be generated by cleavage of a whole antibody using the enzyme papain, thereby obtaining an intact light chain and part of a heavy chain;
(2) the Fab′ fragment of an antibody molecule can be produced by treatment of a whole antibody with pepsin and subsequent reduction, thereby obtaining an intact light chain and part of a heavy chain; two Fab′ fragments per antibody molecule are obtained;
(3) F(ab′)₂, the fragment of the antibody, which can be obtained by treatment of a whole antibody with the enzyme pepsin without subsequent reduction; F(ab′)₂ is a dimer comprised of two Fab′ fragments held together by two disulfide bonds;
   (4) Fv, defined as a fragment modified by genetic engineering, which includes the variable region of the light chain and the variable region of the heavy chain and is expressed in the form of two chains; and
   (5) single-chain antibody (ScA), defined as a molecule modified by genetic engineering, which includes the variable region of the light chain and the variable region of the heavy chain linked by a suitable polypeptide linker to form a genetically fused single-chain molecule.

Methods of producing these fragments are well-known in the art (cf. e.g. Harlow and Lane, Antibodies: A Laboratory Manual, (1988), Cold Spring Harbor Laboratory, New York).

The term epitope as used in the present invention represents any antigen determinant on antigens, particularly those associated with L1, L1-associated molecules such as sugar residues or proteins regulated by an L1 promoter, to which the paratope of an antibody binds. Epitope determinants normally consist of chemically active surface groups of molecules such as amino acids or sugar side-chains and normally have specific features of the three-dimensional structure, as well as specific charge properties.

Those skilled in the art can easily produce monoclonal antibodies in the meaning of the invention, directed against the inventive proteins and fragments thereof or other biological structures. General methods of producing Monoclonal antibodies using hybridoma techniques are well-known. Immortalized cell lines producing antibodies can be generated using cell fusion as well as other methods such as direct transformation of B lymphocytes with oncogenic DNA or transfection with Epstein-Barr virus. See, for example, M. Schreier et al., “Hybridoma Techniques” (1980); Hammerling et al., “Monoclonal Antibodies and T-cell Hybridomas” (1981); Kennet et al., “Monoclonal Antibodies” (1980); see also U.S. Pat. Nos. 4,341,761, 4,399,121, 4,427,783, 4,444,887, 4,452,570, 4,466,917, 4,472,500, 4,491,632, and 4,493,890. Groups of monoclonal antibodies or fragments thereof directed against the protein of interest can be screened with respect to various properties, namely, isotope, epitope, affinity, etc. In another embodiment, genes encoding monoclonal antibodies of interest can be isolated from hybridomas using PCR methods well-known in the art, cloned into suitable vectors, and expressed. When using immunoaffinity methods, monoclonal antibodies are suited to purify the individual cell comprising proteins, such as L1 equivalents, which they are directed against. Regardless whether monoclonal or polyclonal antibodies are concerned, the antibodies according to the invention are useful in that they can be used as reagents in immunoassays such as RIA, ELISA and the like. Furthermore, they can be used in the isolation of the L1 equivalents or of domains from cells or of other biological samples. For example, the antibodies can be used to establish an assay based on a tissue culture to find, isolate or modify new L1 antigens or new compounds that modify the interaction between L1 antigens and receptors and/or target sites.

Humanized or chimeric antibodies may include portions derived from two different species (e.g. human constant region and mouse binding region). The portions derived from two different species can be chemically linked using conventional methods, or can be produced as a single fusion protein using methods of genetic engineering. A DNA encoding the proteins of both portions of the chimeric antibody can be expressed as a single fusion protein.

An antibody binds specifically to a protein, e.g. another biological structure, or exhibits specific immunoreactivity in this way if the antibody assumes its function in a binding reaction in the presence of a heterogeneous population of proteins and other biological substances, which reaction allows a decision to be made whether the protein or another biological structure is present and, in particular, whether the cells comprising the proteins can be isolated. Under the established conditions of an immunoassay, the antibodies mentioned preferably bind to a specific protein, whereas no significant binding to other proteins present in the sample takes place. Specific binding to a protein under such conditions requires an antibody which has been selected according to its specificity for a particular protein, such as L1. Various variants of immunoassays can be used to select antibodies having specific immunoreactivity with a particular protein. For example, solid-phase ELISA immunoassays are routinely used to select monoclonal antibodies having specific immunoreactivity with a particular protein. A description of immunoassay variants and conditions which can be used to determine a specific immunoreactivity can be found in Harlow and Lane, Antibodies; A Laboratory Manual (1988), Cold Spring Harbor Publications, New York.

Preferably, the antibody is a single-chain antibody, a multi-body, a Fab fragment, an MHC molecule, an MHC peptide, a fusion peptide, a mimicry peptide single-chain antibody imitating a conformational epitope, a polyclonal, a monoclonal, a humanized and/or a labelled antibody.

In a preferred embodiment of the invention the neurons are afferent, efferent, intercalary, peripheral-motoric, central-motoric, preganglionic, postganglionic, and/or sensitive neurons.

The invention also relates to a method of isolating neurons, neural and/or neuronal stem cells from a sample, wherein a recognition molecule directed towards a protein regulated by an L1 promoter, towards an L1-associated molecule and/or towards L1 is contacted with the sample, thereby forming recognition molecule-L1 association products, and the association products are separated. The same definitions as in the above-mentioned use apply to the method according to the invention, so that reference can be made thereto. The recognition molecules directed towards the protein regulated by an L1 promoter, towards the L1-associated molecule and/or towards L1 form association products with the above-mentioned structures. In contrast, no association products will be formed with structures, especially cells, included in the sample, which do not have the above-mentioned L1 equivalents. The different charge, density or size of those cells not forming any association products and those representing a component of the association products allows separation of these different cells. Various methods for this purpose are well-known to those skilled in the art. The method of the invention can be preceded or followed by several mechanical, physical, chemical and/or biological steps; for example, the sample can be broken up first using enzymes or simple mechanical size reduction or specific forms of ultrasonic treatment to disintegrate the more or less intact tissue as a whole, without destroying the individual cells, particularly the neurons to be isolated or the above-mentioned stem cells.

Thus, the method results in association products which can be separated, thereby obtaining the isolated cells. In particular, it is possible to separate the cells from the recognition molecules in a further step wherein the association products are disintegrated. Where the recognition molecules are immobilized on a support, the isolated cells can be removed therefrom using an enzyme. However, it can also be envisaged not to separate the cells from the recognition molecules, e.g. chelators, particularly if these molecules have a favorable influence on the physiology of the cells consequently, the separated association product can be an isolated cell in the meaning of the invention, as is the case with the cell which is no longer part of an association product because it has been separated from the recognition molecules.

Various standard publications on histology, tissue or cell culture technology are known to those skilled in the art, from which it can be inferred how to pretreat the sample in accordance with the cells included therein, in order to disintegrate the whole sample, e.g. an embryonal mouse brain, in a way so as to allow separation of cells. For example, initial leaching of calcium from the tissue can be envisaged, so as to break up the cell linkages. Depending on the type of samples, initial introduction of calcium into the tissue may also be reasonable in achieving the effect of breaking up the cell linkages.

In a further step, adapting the extracellular solution in its ion content to the intracellular solution can be reasonable or advantageous, so that the cells would be in a better situation to survive in particular small lesions during the isolation procedure. Depending on the cells to be obtained, it is of course also possible not to adapt the extracellular solution in its ion content to the intracellular solution.

Furthermore, perfusion of enzymes breaking up the cell-cell linkages and disintegrating the extracellular collagen framework possibly stabilizing the tissue can also be envisaged. To this end, e.g. the successive use of enzymes such as trypsin and collagenase in a solution including a high proportion of potassium is advantageous. This step can be omitted if the sample contains no or merely an insignificant collagen framework, or if treatment with enzymes such as trypsin and/or collagenase of the cells to be isolated is undesirable, or if a high proportion of potassium is undesirable. However, it is also possible to envisage an enzyme treatment using a solution including a proportion of other uni-, bi- or polyvalent elements, particularly metal ions, rather than a solution including a high proportion of potassium.

In a further step, the broken up tissue can be cut and single cells can be removed from the tissue network by mechanical stirring. At the same time, it is possible e.g. to re-transfer the cells to be isolated into an ionic solution of normal composition, the term normal composition implying that the solution ensures that pathological conditions would not prevail over physiological conditions in the cells to be isolated, thereby enabling further culturing or growth or differentiation of the cells.

In a further step, pre-separation of the cells via centrifugation, sedimentation or other purification steps can be advantageous.

For example, further purification can be effected in chromatography columns, in a cell electrophoresis, in a FACS apparatus, or by means of other methods of cell purification and cell isolation. However, these steps can also be performed in the method according to the invention rather than in a previous procedure.

In chromatography, such as a column-chromatographic separation, it is possible to use nylon wool, for example, on which the recognition molecules are immobilized, so that the cells to be isolated interact with the wool in such a way that isolation is possible.

Furthermore, the cells can also be separated using centrifugation, for example. More specifically, the cells can be separated using a rotating centrifuge, the cells being introduced via so-called bubble filling obviously, separation can also be effected in centrifuge tubes as commonly used. Similarly, any procedure used in cell separation, e.g. in the preparation of concentrates, can be employed according to the invention. This may also include cell separation by means of magnetic beads. That is, all separation methods based on differences in the cell density, e.g. isopyknic centrifugation, the cell size, e.g. elutriation centrifugation, the adherence properties, e.g. adsorption on plastic or nylon wool fractionation, can be employed.

In addition, separation involving magnetic particles can be used in cell separation or isolation.

For example, such a separation can be effected using MACS or Mini-MACS or particular FACS apparatus.

Of course, all of the above-mentioned procedures can be combined with one another, i.e., it is possible to combine e.g. magnetic bead separation with flow cytometry. However, successively employed recognition molecules may not recognize the same epitope in each case, thereby giving rise to competition. Various ways of avoiding this problem or others are well-known to those skilled in the art.

Afferent, efferent, intercalary, peripheral-motoric, central-motoric, preganglionic, postganglionic neurons are isolated as neurons in a preferred embodiment of the invention.

In another preferred embodiment the neurons are adendritic, apolar, bipolar, multipolar, polyneuritic, pseudo-unipolar, unipolar and/or neurosecretory neurons.

A brain, a striatum, a neocortex, a spinal cord and/or a partial quantity thereof is employed as sample in another preferred embodiment of the invention.

Furthermore, stem cells, in particular differentiated stem cells, are preferably used as sample, the use of neural and/or neuronal stem cells being particularly preferred.

In a particularly preferred embodiment the sample comprises a neural-differentiated embryonal and/or a neural-differentiated adult stem cell.

In another preferred embodiment of the invention molecules selected from the group comprising an antibody, an aptamer, a lectin, an antisense construct, a selective chelator, fragments or a combination thereof are employed as recognition molecules.

In a particularly preferred fashion an antibody fragment, a single-chain antibody, a multibody, a Fab fragment, an MHC molecule, an MHC peptide, a fusion peptide, a mimicry peptide single-chain antibody imitating a conformational epitope, a polyclonal, a monoclonal, a humanized and/or labelled antibody is used as antibody in the method according to the invention.

In another preferred embodiment of the invention the recognition molecules are immobilized. Immobilization in the meaning of the invention is understood to comprise all methods restricting the mobility and solubility of recognition molecules or L1 equivalents by chemical, biological and/or physical means. Immobilization can be effected using various methods, such as binding of recognition molecules to one another, or to culture dishes or separation surfaces, e.g. nylon wool, by entrapping in a network of a polymer separation matrix or by enclosure by membranes. Immobilization not only renders the recognition molecules reusable but also, they can easily be removed after the process of interaction with the biological sample. They can be employed at much higher local concentrations and in continuous flow systems. Binding or immobilization of the recognition molecules on a surface can be effected by direct linkage to a support, in particular mediated via spacer binding and crosslinking. According to the invention, support binding or crosslinking is effected particularly in an ionic/adsorptive fashion or by covalent binding. Crosslinking in the meaning of the invention is crosslinking of recognition molecules with each other or with other polymers. In immobilization by enclosure the recognition molecules are entrapped in gel structures or in membranes in such a way that interaction and isolation of the desired cells is possible.

In another preferred embodiment of the invention the recognition molecules are immobilized by physical, chemical and/or biological means, by in situ synthesis, or by deposition of previously synthesized recognition molecules.

In an advantageous embodiment of the binding assay the recognition molecules are immobilized by physical, chemical and/or biological means, by in situ synthesis, or by deposition of previously synthesized recognition molecules on a surface. Irrespective of the type of immobilized recognition molecules, this can be done basically in two different ways:
1. Deposition and immobilization of previously synthesized or library-derived recognition molecules on well-defined positions of an, in particular, functionalized support material. To this end, both spotting and printing procedure can be used. Spotting is understood to comprise procedures wherein drops of a liquid are deposited, essentially round spots being formed as a result of surface interaction and drying. Other printing procedures allow deposition of the substrate in well-defined areas on the surface.
2. In situ synthesis of recognition molecules at defined positions of a surface, e.g. on a culture vessel or on a chromatographic support material, by successive coupling of monomeric components for synthesis.

In another advantageous embodiment the recognition molecules are immobilized by contact tip printing, ring-and-pin printing and nanopipetting, bubble-jet printing, top-spot printing, microcontact printing, micro-fluidic networks methods, photolithographic activation procedures, photoresist lithography, electrochemical focusing and/or micro-wet printing.

In another preferred embodiment of the invention the surface, e.g. of the culture or washing vessels or other equipment or materials used in separation, is coated with poly-L-lysines, aminosilanes, aldehyde-silanes, epoxy groups, streptavidin, polylysines, silanes, reactive groups, polyacrylamide pads, immobilized nitrocellulose, activated aldehydes, agarose aldehyde groups and/or tresyl groups. Advantageously, such substrate surface treatment allows improvement of the binding capacity of the surface in such a way that the recognition molecules remain properly immobilized over a prolonged period of time. Obviously, it is also possible to treat the surface in such a way that the cells to be isolated have good adherence and the other components have less good adherence, or vice versa. Of course, surface modification can be effected in a broad fashion, and particularly by exposure to biological, physical and/or chemical effects. For example, physical exposure includes polishing, etching, scouring, sandblasting, but also, physical procedures resulting in curing, coating, finishing, coating with protective skins and the like. Surface treatment by biological exposure may comprise growth of selected cells, for example. Chemical modification of the surface involves e.g. treatment with acids, bases, metal oxides etc. The surface can be modified in such a way that the detection molecules or recognition molecules have particularly good adherence, or adherence in such a way that their activity will not be adversely modified. Naturally, surface modification of the separation materials or culture vessels also comprises a treatment resulting in higher stability or improved culture conditions. Obviously, it is also possible to perform traditional surface modifications as in histology, such as coating with e.g. protein glycerol, polylysine, activated dextranes and/or bichromated gelatin.

In another preferred embodiment of the invention immune complexes are formed as association products. More specifically, immune complexes are formed at an optimum concentration ratio between L1, L1-associated molecule or protein regulated by an L1 promoter and the recognition molecules. In the meaning of the invention it is possible to distinguish between soluble and circulating immune complexes.

In a preferred embodiment of the invention the immune complexes are antibody-antigen complexes or lectin-antigen complexes.

In the meaning of the invention the antigen is the L1 cell adhesion protein, the L1-associated molecule and/or the protein regulated by an L1 promoter, or the L1 equivalents.

In particular, lectins can be structures undergoing highly specific interaction with molecules associated with L1 in such a way that isolation of the desired cells is possible. For example, such lectins can be isolated from the following organisms: Abrus precatorius, Adenia digitata, Agaricus bisporus, Aleuria aurantia, Amaranthus caudatus, Amphicarpaea biacteata, Anguilla anguilla, Arachis hypogaea, Artocarpus integrifolia, Bauhinia purpurea alba, Brachypodium sylvaticum, Canavalia ensiformis, Carcino scorpius rotunda canda, Cicer arietinum, Codium fragile, Crotalaria juncea, Cytisus sessilifolius, Datura stramonium, Dioclea grandiflora, Dolichos biflorus, Erythrina coralldendron, Erythrina cristagalli, Euonymos europaea, Galanthus nivalis, Glycine max, Griffonia (Bandeiraea) simplicifolia, Helix aspersa, Helix pomatia, Hippeastrum hybrid, Hordeum vulgare, Hura crepitaus, Latyrus odoratus, Latyrus sativus, Latyrus tingitanus, Lens culinaris, Limax glavus, Limulus polyphemus, Lotus tetragonolobus, Lycopersicon esculentum, Maackia amurensis, Maclura pomifera, Macrotyloma axillare, Momordica charantia, Narcissus pseudonarcissus, Oryza sativa, Phaseolus coccineus, Phaseolus limensis, Phaseolus vulgaris, Phytolacca americana, Pisum sativum, Phosphocarpus tetragonolobus, Pseudomonas aeruginosa, Ricinus communis, Sambucus nigra, Secale cereale, Solanum tuberosum, Sophora japonica, Triticum vulgaris, Tritrichomonas mobilensis (Protozoa), Ulex europaeus, Vicia cracca, vicia ervilia, Vicia graminea, Vicia faba, Vicia sativa, Vicia villosa, Viscum album, Wisteria floribunda, and also, the following human lectins, particularly produced on a recombinant route, can be used: E-selectin (human, recombinant) L-selectin (human, recombinant) and/or P-selectin (human, recombinant), as well as galectins (human).

The antibodies can be obtained from any organism which, as a result of dealing with antigenic immunologic recognition substances, is capable of forming in particular antibodies directed towards the latter.

In another preferred embodiment of the invention the association products are antisense hybrids, with DNA-DNA, RNA-RNA and/or RNA-DNA antisense hybrids being preferred. Hybridization in the meaning of the invention is sequence-dependent pairing of single-stranded RNA or DNA molecules to form an RNA/DNA hybrid. The term antisense symbolizes the complementary structure of each single strand with respect to another single strand. A variety of antisense techniques are known to those skilled in the art, particularly in the field of electrochemical detection of oligonucleotides. A person skilled in the art will also know how to employ these antisense constructs, e.g. in immobilized form, in order to use them in the isolation of the desired cells. In this event, the antisense constructs would serve as so-called scavenger sequences which are bound to a surface, for example, e.g. a culture dish, and are complementary to part of a nucleic acid structure encoding an. L1-associated molecule, a protein regulated by an L1 promoter, or an L1. For example, it can also be envisaged to make initial use of the antisense constructs to detect the corresponding structures and use them Subsequently on a protein level, e.g. together with antibodies, to isolate the desired cells.

In another preferred embodiment of the invention separation of the desired cells is effected via different density, size and/or charge of the neurons, of the neuronal and/or neural stem cells, being present in the form of association products, and of components of the samples not containing any association products. The term “not containing any association products” refers to components of a sample which essentially do not comprise any L1 cell adhesion proteins, L1-associated molecules and/or proteins regulated by an L1 promoter, or exhibit concentrations of these structures at such low levels that isolation via use thereof is not possible. However, those components of a sample which bear these structures will form L1-recognition molecule association products with the recognition sequences, the sample including association products as well as proteins, cells, tissue fragments and other biological units lacking such association products. The above two groups differ in their density, size and/or charge.

In a preferred embodiment of the invention said association products are separated or isolated using affinity chromatography, cytolysis, FACS, density gradient separation, adhesion, agglutination, rosette formation and/or cell electrophoresis. With reference to a non-limiting example, this principle will be illustrated as follows: for example, it can be envisaged that the recognition molecules are antibodies immobilized on a Petri dish. A cell suspension including neurons, among other things, can be placed on this Petri dish. For example, these neurons can be isolated on the Petri dish as so-called L1-positive cells via formation of association products. However, it can also be envisaged to use the recognition molecules in the form of a column material, e.g. in a chromatographic column, or to bind the recognition molecules to such a column material in order to separate a cell suspension by means of said column material, in which case the L1-positive cells will adhere to the column material and all non-L1-positive cells will pass through the column. The bound cells are then removed from the column material using specific elution procedures.

In the meaning of the invention L1-positive cells are all cells having L1 or L1-associated molecules or proteins regulated by an L1 promoter at a concentration or with a structure allowing isolation of neurons, neural and/or neuronal stem cells from a sample via use thereof. Those skilled in the art are familiar with other methods of cell separation, e.g. cell separation using magnetic beads or the “panning” procedure or FACS, adsorption on plastic, nylon wool fractionation, various forms of centrifugation utilizing the cell size or cell density as distinguishing feature of the cells present in the form of association products or non-associated. In magnetic bead cell separation the L1 equivalents serve as surface markers, the specific recognition molecules being used in the form of magnetic bead-coupled recognition molecules.

In another preferred embodiment the sample is initially pretreated with an SH-proteinase. In a preferred fashion said SH-proteinase is papain.

In another preferred embodiment initial depletion of microglia cells in the sample is envisaged. For example, such depletion can be effected—using one, two or more subtraction plates—by adding the suspension with e.g. anti-macrophage antibodies and successively placing the suspension on two dishes coated with secondary antibodies. The microglia cells, for example, will adhere to the dishes because they bind to Fc fragments of the antibodies.

In another preferred embodiment removal with trypsin of the isolated neurons, neural and/or neuronal stem cells from the immobilized recognition molecules or from the surfaces of the culture or separation materials is envisaged. For example, L1-positive cells are removed from anti-L1 antibody-coated culture dishes by treatment with trypsin.

According to another embodiment of the invention, the cells thus isolated are then plated on cell culture dishes coated with poly-D-lysine and/or laminin.

The invention also relates to purified neuron, neural and/or neuronal stem cell populations, particularly those obtained by means of the method according to the invention; accordingly, the invention also relates to cell populations having appropriate degrees of purity, which can be obtained in another way. Preferred are those cell populations with a purity of more than 90%. That is, the cells are present without the material associated with the cells in their natural state or with part thereof at maximum. Based on the weight or volume of the total cells in a particular sample, the purified cells account for at least 90%. In a particularly preferred fashion the isolated cells are essentially free of other cells, but also of proteins, lipids, carbohydrates, or other substances associated with the cells in such a way that a person skilled in the art would not regard them as cells of high purity or as isolated cells. Of course, this does not mean that cells could not be associated with proteins, lipids, carbohydrates, particularly where molecules are concerned that are synthesized by the neurons, neural and/or neuronal stem cells themselves. “Essentially isolated” or “essentially free of” means that the cells are at least 90%, preferably at least 95%, preferably at least 97%, more preferably at least 98%, and especially preferably more than 99% free of other cells naturally associated therewith.

The invention also relates to the above-mentioned cell populations with high purity.

In a preferred embodiment of the invention the cells are available with a purity of 95%. In another, particularly preferred embodiment of the invention the cells are available with a purity of more than 97%. In another particularly preferred embodiment of the invention the cells are available with a purity of more than 98%, and in yet another particularly preferred embodiment of the invention the cells are available with a purity of more than 99%.

The invention also relates to a purification kit comprising a recognition molecule directed towards a protein regulated by an L1 promoter, towards an L1-associated molecule and/or towards an L1 cell adhesion protein. Using said kit, a person skilled in the art is able to isolate the above-mentioned cells which are to be purified. Isolation is preferably performed using the method described above. Owing to the teaching that has been provided, a person skilled in the art can isolate the cells using routine tests or equivalent uses, said routine procedures and equivalents being hereby incorporated in the teaching of the present patent application.

The invention also relates to a microarray comprising a recognition molecule directed towards a protein regulated by an L1 promoter, towards an L1-associated molecule and/or towards an L1 cell adhesion protein.

Without intending to be limiting, the invention will be explained in more detail with reference to the following examples.

An immunopanning procedure was established, allowing purification of neurons from hippocampi of postnatal mice via the neuron-specific L1 antigen (Rathjen & Schachner, 1984). FIG. 1 illustrates the procedure for the preparation of hippocampus neurons. Initially, a suspension of dissociated hippocampi was depleted in microglia cells, using two subtraction plates. Thereafter, L1-positive cells were isolated on a selection plate. Immunopanning via L1 allowed isolation of an average of 294,000±37,000 cells (n=11 work-ups) from the hippocampi of 10-15 postnatal mice (day 6-7).

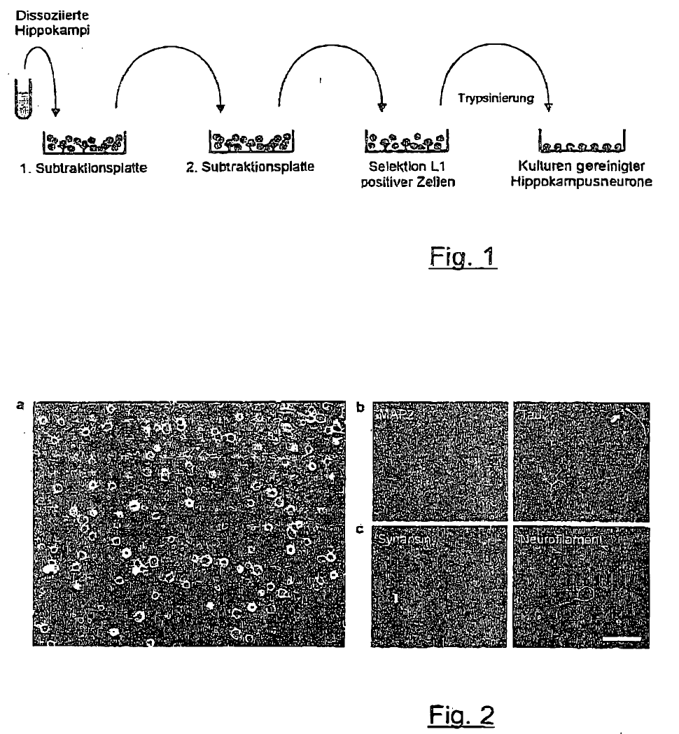

Using said immunopanning, it was possible to isolate a large number of cells. Now, it was important to see whether these cells were neurons and whether the cultures would also contain non-neuronal cells. After 2 days in culture, all cells showed neuron-like morphology and immunoreactivity to antibodies against the neuron markers tau, MAP2, neurofilament, and synapsin (FIG. 2). Moreover, the cells had axonal growth cones. Differentiation of neurites into axons can be derived from the fact that some appendices could be stained for the tau axon marker, but not for the marker of dendrites, MAP2 (FIG. 2a). Furthermore, some growth cones had synapsin-positive granules typical of growth cones of axons (FIG. 2b). These results demonstrate that the isolated cells were neurons. It was possible to show that 17±2% (mean value±standard error; n=4 work-ups) of all neurons contained in the suspension of dissociated hippocampi could be isolated on the selection plate.

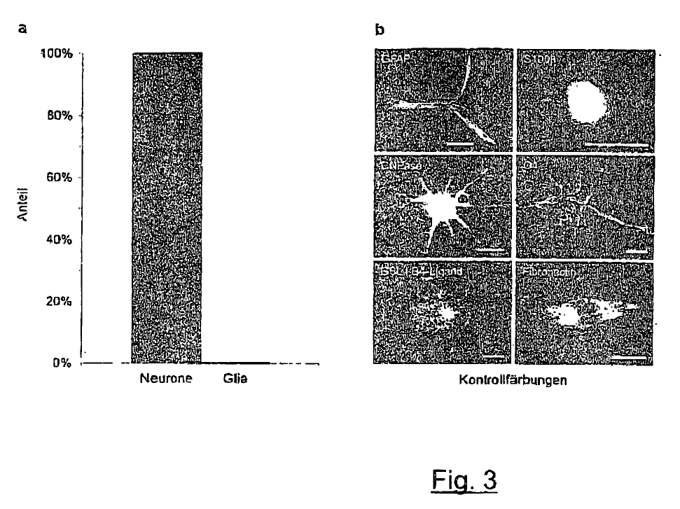

Next, the cultures were investigated for the presence of cell types other than neurons. To this end, immunocytochemical staining for markers of a number of non-neuronal cell types was performed (FIG. 3). The macroglia cells were detected by staining for the specific markers 2′,3′-cyclonucleotide-3′-phosphohydrolase (CNPase) and O4 for oligodendrocytes and precursor cells thereof, and for glial fibrillary acidic protein (GFAP) for astrocytes. Since not all of the astrocytes of the hippocampus express GFAP, staining was also performed for S100β which is another astrocyte marker. Fibroblasts were detected via fibronectin, microglia cells via Griffonia simplicifolia lectin I isolectin B4 (GSL I-B4). Control experiments showed that these non-neuronal cell types could be detected reliably by means of the employed markers in one to two-day-old control cultures containing a mixed cell population of dissociated hippocampi from mice (FIG. 3b). Cultures of neurons isolated via L1 immunopanning did not include any astrocytes. Oligodendrocytes were detected in very low amounts of <0.1% (n=4 cultures; FIG. 3a). Also, the cultures were free of fibroblasts or microglia cells (n 3 cultures, 1363 cells).

These results show that cell populations comprising >99.9% of hippocampus neurons can be obtained by means of the new immunopanning procedure.

Also, cell isolation via L1 was found suitable in the purification of neural-differentiated embryonal stem cells for neurons. Fibers growing out of embryoid bodies (EBs) were shown to express L1 after differentiation by retinolic acid. Using immunopanning of dissociated EBs, it was possible to isolate a large number of cells which showed neuron-like morphology and immunoreactivity to the MAP2 neuron marker after culturing for one week. Only aminor proportion of the cells (<99%) did not have appendices and/or failed to express MAP2.

In summary, it can be stated that the method according to the invention allows highly effective isolation of neurons from mixed cell populations. Using this method, it is possible to accumulate neurons with high purity either from postnatal brain tissue or from neural-differentiated stem cells. Neurons purified in this fashion are suitable for use in investigating the conditions of neuronal growth and differentiation. Compared to mixed cell populations, they also offer advantages in transplantations for the treatment of neurodegenerative diseases.

FIG. 1 Purification of Hippocampus Neurons

Hippocampi from 10-15 postnatal mice (day 6-7) were pretreated with papain, followed by mechanical dissociation. Initially, the cell suspension was depleted in microglia cells by adding the suspension with e.g. anti-macrophage antibodies and successively placing the suspension on two dishes coated with secondary antibodies. In this step the microglia cells—binding to the Fc fragments of the antibodies—remained adhered to the dishes. Subsequently, L1-positive cells were isolated from the cell suspension, using a dish coated with secondary antibodies and anti-L1 antibodies. The cells were removed from the dish by treatment with trypsin and subsequently plated on cell culture dishes coated with poly-D-lysine and laminin.

FIG. 2 Neuron-Like Morphology and Immunoreactivity to Antibodies Against Neuronal Markers in Isolated Hippocampus Cells

a Phase contrast image of a two-day-old culture. The cells already have neurite-like appendices with growth cones.

b, c Immunocytochemical double-labelling for neuron markers MAP2 and tau (a) and neurofilament and synapsin (b). In stained cultures all cells recognizable in the phase contrast were positive for the respective neuron marker. The arrows denote axonal growth cones. Bar=10 μm (a), 40 μm (b, c)

FIG. 3 Purity of Cultures of Isolated Hippocampus Neurons

a The proportion of macroglia cells and neurons relative to the overall number of cells identified via fluorescence-labelled nuclei is illustrated. To detect macroglia cells, two-day-old cultures of purified hippocampus neurons were simultaneously stained immunocytochemically with antibodies against GFAP, S100β, O4 and CNPase. Two out of 2451 cells (n=4 cultures) were identified as oligodendrocytes. The error bars represent standard deviations.

b Control stainings showed that the markers used reliably detect the respective cell types in two-day-old mixed cultures of dissociated mouse hippocampi. Bar 20 μm.

1-36. (canceled)

37. Use of an L1 cell adhesion protein for the isolation of neurons, neural and/or neuronal stem cells from a sample.

38. The use according to claim 37,
characterized in that
the neurons are afferent, efferent, intercalary, peripheral-motoric, central-motoric, preganglionic, post-ganglionic, and/or sensitive neurons.

39. A method for the isolation of neurons, neural and/or neuronal stem cells from a sample,
characterized in that
an antibody directed against L1 is contacted with the sample, thereby forming antibody-L1 association products, and the association products are separated.

40. The method according to claim 39,
characterized in that
afferent, efferent, intercalary, peripheral-motoric, central-motoric, preganglionic, postganglionic, and/or sensitive neurons are isolated as neurons.

41. The method according to claim 40,
characterized in that
the neurons are adendritic, apolar, bipolar, multipolar, polyneuritic, pseudo-unipolar, unipolar and/or neurosecretory neurons.

42. The method according to claim 39,
characterized in tht
a brain, a striatum, a neocortex, a spinal cord and/or a partial quantity thereof is employed as sample.

43. The method according to claim 39,
characterized in that
stem cells are used as sample.

44. The method according to claim 43,
characterized in that
neural and/or neuronal stem cells are used as sample.

45. The method according to claim 44,
characterized in that
neural-differentiated embryonal and/or neural-differentiated adult stem cells are used as sample.

46. The method according to claim 39,
characterized in that
an antibody fragment, a single-chain antibody, a multi-body, a Fab fragment, an MHC molecule, an MHC peptide, a fusion peptide, a mimicry peptide single-chain antibody imitating a conformational epitope, a polyclonal, a monoclonal, a humanized and/or labeled antibody is used as antibody.

47. The method according to claim 37,
characterized in that
the antibodies are immobilized.

48. The method according to claim 47,
characterized in that
the antibodies are immobilized by physical, chemical and/or biological means, by in situ synthesis, or by deposition of previously synthesized antibodies.

49. The method according to claim 48,
characterized in that
the antibodies are immobilized by contact tip printing, ring-and-in printing, nanopipetting, bubble-jet printing, top-spot printing, microcontact printing, microfluidic networks methods, photolithographic activation procedures, photoresist lithography, electrochemical focusing and/or micro-wet printing.

50. The method according to claim 48,
characterized in that
an immobilization surface is coated with poly-L-lysines, aminosilanes, aldehyde-silanes, epoxy groups, streptavidin, reactive groups, polyacrylamide pads, immobilized nitrocellulose, activated aldehydes, agarose aldehyde groups and/or tresyl groups.

51. The method according to claim 39,
characterized in that
immune complexes are formed as association products.

52. The method according to claim 37,
characterized in that
separation is effected via different density, size and/or charge of the association products of components of the sample not comprising any association products.

53. The method according to claim 52,
characterized in that
separation is effected using affinity chromatography, cytolysis, FACs, density gradient separation, adhesion, agglutination, rosette formation and/or cell electrophoresis.

54. The method according to claim 37,
characterized in that
the sample is pretreated with an SH-proteinase.

55. The method according to claim 54,
characterized in that
the SH-proteinase is papain.

56. The method according to claim 37,
characterized in that
the sample is initially depleted in microglia cells.

57. The method according to claim 37,
characterized in that
the isolated neurons, neural and/or neuronal stem cells are removed from the immobilized antibodies by means of trypsin.

58. The method according to claim 37,
characterized in that
the isolated neurons, neural and/or neuronal stem cells are plated on cell culture dishes coated with poly-D-lysine and/or laminin.

59. Neurons, neural and/or neuronal stem cell population which can be obtained by means of a method according to claim 39.

60. The neurons, neural and/or neuronal stem cell population according to claim 59,
characterized in that
the cells are available with a purity of more than 90%.

61. The neurons, neural and/or neuronal stem cell population according to claim 60,
characterized in that
the cells are available with a purity of more than 95%.

62. The neurons, neural and/or neuronal stem cell population according to claim 61,
characterized in that
the cells are available with a purity of more than 97%.

63. The neurons, neural and/or neuronal stem cell population according to claim 62,
characterized in that
the cells are available with a purity of more than 98%.

64. The neurons, neural and/or neuronal stem cell population according to claim 63,
characterized in that
the cells are available with a purity of more than 99%.

65. A purification kit comprising an antibody directed against L1.

66. A microarray comprising an antibody directed against L1.
